(19) United States
(12) Patent Application Publication
(10) Pub. No.: US 2006/0127324 A1
(43) Pub. Date: Jun. 15, 2006

(54) Chewable oral compositions containing an anti-microbial agent and methods of formulating the compositions to have acceptable germ kill properties and taste

(75) Inventor: Shiuh John Luo, Livingston, NJ (US)

Correspondence Address: Allen R. Kipnes, Esq.;WATOV & KIPNES, P.C., P.O. Box 247, Princetion Junction, NJ, 08550, US

(73) Assignee: Cadbury Adams USA LLC

(21) Appl. No.: 11/010,082

(22) Filed: Dec. 10, 2004

(51) Int. Cl. A61K 9/68; G06F 19/00; A61K 36/54; A61K 36/61; A61K 36/534

(52) U.S. Cl. 424/48; 702/19; 424/747; 424/739; 424/742

(57) ABSTRACT

A method of formulating anti-microbial containing oral compositions having a targeted (P) value by selecting at least one anti-microbial agent based on the germ kill rating (R) and concentration of the anti-microbial based on the agent, the mass of the oral composition and the Q factor of the oral composition.

### FIELD OF THE INVENTION

The present invention is generally directed to a consumable oral compositions containing anti-microbial agents and, and more particularly to compositions which can be formulated to deliver effective germ kill properties while exhibiting pleasant taste characteristics.

### BACKGROUND OF THE INVENTION

Oral diseases including dental plaque, gingivitis, tooth decay and oral malodor are caused by the undesirable presence of pathogenic microorganisms in the mouth. A regular oral cleaning regimen can offer an effective check on the presence of such microorganisms and at least reduce the incidence of oral disease. Many oral cleaning formulations have been developed to promote thorough cleansing and disinfecting of the oral cavity. Some formulations including mouth rinses contain anti-microbial agents, including, but not limited to, essential oils such as thymol, menthol, methyl salicylate and eucalyptol, which possess potent anti-microbial activity to enhance the cleansing action. Essential oils are the most commonly used anti-microbial agents in dental hygiene products.

It is difficult to prepare such compositions because anti-microbial agents such as essential oils require a minimum effective amount to provide acceptable germ kill properties, but at the same time exhibit unpleasant taste characteristics which deter the incorporation of elevated concentrations of the anti-microbial agents in an oral composition. Because of the unpleasant taste, anti-microbial oral compositions are often limited to mouthwashes which are retained in the oral cavity for short periods of time. Thus, one response to the unpleasant taste characteristics is to limit the retention time of the oral composition in the oral cavity. However, a short retention time either does not provide sufficient time for the anti-microbial agent to adversely affect microorganisms contained in the oral cavity or requires that the oral composition contain high concentrations of the unpleasant tasting anti-microbial agents. Accordingly, it has been difficult to formulate oral compositions containing anti-microbial agents which possess effective germ kill properties and yet also possess desirable taste characteristics.

Although it is known to use elevated concentrations of anti-microbial agents such as essential oils in mouthwashes, such compositions are not typically fit for consumption and thus have to be expectorated from the mouth after being retained in the oral cavity for a short period of time. For this reason, such formulations are often inconvenient to use especially outside the home where personal care facilities are often not available.

Another approach to solving this problem is to employ high concentrations of sweeteners, flavorants and/or other taste-masking agents. However, this approach to overcoming the problem of unpleasant taste is often inadequate because the taste-masking agents may need to be used in elevated concentrations wherein the taste of the composition is adversely affected.

The incorporation of anti-microbial agents into oral compositions such as chewing gums and confectionery products is problematic for the reasons discussed above. The harsh taste of anti-microbial agents makes it difficult to formulate an oral composition that a consumer is expected to chew or ingest and retain in the oral cavity for an extended period of time. Unlike mouthwashes which can be expectorated after only a short period of time, consumable products such as chewing gums and confectionery compositions are often retained in an oral cavity for extended periods of time adding to the difficulty of dealing with effective germ kill properties and desirable taste characteristics.

As previously indicated, taste masking agents typically do not solve the problem or provide the necessary relief from the harsh taste of the anti-microbial agents. If the concentration of these taste masking agents is elevated, the taste masking agents themselves can provide an unacceptable taste characteristic to the product. On the other hand, if the concentration of the anti-microbial agent is reduced to alleviate the problem of unpleasant taste, the concentration of the anti-microbial agent is often insufficient to provide adequate germ kill properties, particularly for products like chewing gums and confectionery products.

It would therefore be a significant advance in the art to provide a method of formulating anti-microbial containing oral compositions that provide both effective germ kill properties and desirable taste characteristics. The method would desirably provide the means by which a wide range of anti-microbial agents could be selected and various characteristics of the composition could be predictably selected to produce an oral composition with predictable effective germ kill properties and desirable taste characteristics, especially chewing gums and confectionery products.

### SUMMARY OF THE INVENTION

The present invention is generally directed to a method of formulating anti-microbial containing oral compositions with acceptable germ kill properties and desirable taste characteristics. The method focuses on identifying a desirable germ kill potential (P) for the oral composition, as hereinafter defined. A suitable anti-microbial agent is selected based on its germ kill rating (as hereinafter defined) and a factor Q which is the percentage of the anti-microbial agent released into the oral cavity after a fixed period of time. A suitable concentration of each anti-microbial agent contained within the composition is selected as is the mass of the product. A suitable range of (P) values can be established for each type of product and the above-mentioned variables selected in a manner which predictably provides a product having acceptable germ kill properties and a desirable taste profile.

In accordance with one aspect of the invention, there is provided a method for formulating an oral composition containing at least one anti-microbial agent, comprising:

establishing a targeted or desired germ kill potential value (P) for the oral composition and selecting at least one anti-microbial agent for the oral composition based on the germ kill rating and the concentration of the anti-microbial agent, the Q factor of the anti-microbial agent in the oral composition and the mass of the oral composition which exhibits the targeted gum kill potential (P), and formulating said oral composition using said at least one anti-microbial agent.

Methods of formulating specific oral compositions for chewing gums, confectionery products and the like as well as products formed by the method are encompassed herein.

In a further aspect of the invention there is provided a method of determining a desired germ kill potential value for an oral composition.

### BRIEF DESCRIPTION OF THE DRAWINGS

The following drawings are illustrative of embodiments of the invention and are not intended to limit the invention as encompassed by the claims forming part of the application.

FIGS. 1 and 2 are three dimensional graphs plotting the germ kill rating of an anti-microbial agent, the concentration of the selected anti-microbial agent and the Q factor of the desired product to arrive at a designated (P) range of values for a hypothetical oral composition having a fixed mass.

### DETAILED DESCRIPTION OF THE INVENTION

The present invention is based on the discovery that it is possible to produce an anti-microbial agent containing oral composition which may be consumed (e.g. placed in the mouth and chewed) or ingested such as a chewing gum composition or a confectionery composition in which the method predictably provides a composition with acceptable germ kill properties and desirable taste characteristics. The method enables the judicious selection of targeted anti-microbial agents and the selection of targeted variables which enable the oral composition to possess a desirable germ kill potential (P) which predictably provides the desirable germ kill properties and taste characteristics.

As used herein, the term “desirable” as it applies to germ kill properties means that the composition kills an effective amount of germs (e.g. bacteria) in the oral cavity to provide an improved environment in the oral cavity for the healthy maintenance of teeth, gums and/or tissues in the oral cavity and elsewhere where the anti-microbial agent may reach. The term “desirable” as it applies to taste characteristics means that the taste of the oral composition is acceptable to a significant portion of humans who ingest the composition.

The term “germ kill potential” as used herein and hereinafter identified as “P” is determined by the following formula

$$P = ( {\sum }_{n = 1}^{Y} {concentration}_{n} \times {R}_{n} ) \times Mass \times Q$$

wherein
Mass=the mass of the composition capable of delivering the anti-microbial agent to the oral cavity,
concentration=the concentration of the anti-microbial agent measured in parts per hundred,
R=the germ kill rating of the anti-microbial agent,
Y=number of anti-microbial agents in the composition, and
Q=is a number obtained by the formula 100/z wherein z is the percentage of the anti-microbial agent effectively released into oral cavity after a fixed time (e.g. 10 minutes of chewing).

The germ kill rating represented by “R” is identified by the following formula:

$$R = \frac{maximum acceptable taste concentration ( ATC )}{minimum inhibitory concentration ( MIC )}$$

wherein
ATC=the maximum concentration of the anti-microbial agent in the solid carrier that still provides an acceptable taste, and
MIC=the concentration of the anti-microbial agent below which bacterial growth in a given medium (e.g. oral cavity) if no longer effectively inhibited.

The germ kill rating (R) is therefore a quantitative indication of the range of concentrations of the anti-microbial agent (i.e. effective concentration range) that may be employed to achieve both the desirable germ kill properties and acceptable taste. By way of example, an anti-microbial agent having a germ kill rating of 10 has an effective concentration range twice as large as an anti-microbial agent having a germ kill rating of 5.

The germ kill potential (P) therefore is determined by the product of the concentration of at least one anti-microbial agent contained in the composition and the germ kill rating of each anti-microbial agent. The result is in turn multiplied by the mass of the composition and a factor Q as defined previously which is a measure of how easily the anti-microbial agent is released from the oral composition into the oral cavity. The Q factor may be readily determined for a given oral composition by comparing the amount of the anti-microbial agent present in the oral composition before placement in the oral cavity and how much remains after placement in the oral cavity for a fixed period of time.

Certain oral compositions such as many confectionery compositions release the anti-microbial agent into the oral cavity instantaneously or soon after it is placed in the oral cavity. The percentage of the anti-microbial agent released into the oral cavity after a fixed period of time (e.g. 10 minutes of chewing) is therefore typically 100% and thus Q would have a value of or about 1.0 [i.e. 100% (amount of anti-microbial agent in the composition)/100% (amount of the anti-microbial agent released into the oral cavity in the fixed period of time)].

For other compositions such as chewing gum compositions, the release rate of the anti-microbial agent is much slower. Typically only a portion of the anti-microbial agent is released instantaneously and/or on initial chewing. For example, the release rate of anti-microbial agents after 10 minutes of chewing can be as low as less than 10%.

The release rate of the anti-microbial agent from the composition is therefore a factor which influences the value of the germ kill potential P. Generally a slow release rate will require a higher (P) value in order to achieve desirable germ kill properties and taste characteristics. By way of example, a menthol containing confectionery composition having a given mass and concentration such as a pressed mint composition will release all of the menthol essentially instantaneously, but certainly within a fixed period of time such as 10 minutes after placement in the oral cavity. The Q value will therefore be 1.

A chewing gum composition of the same mass containing menthol in the same concentration shows a release rate of menthol of about 9% after 10 minutes. Thus, the factor Q is 11.1 (100%/9%). The targeted (P) value selected for the chewing gum composition would therefore have to be about 11.1 times greater than the (P) value for the corresponding pressed mint composition.

Release rates (Q values) of exemplary anti-microbial agents in chewing gum compositions are shown in Table I.

TABLE I

| | Release Rate (%) After |
|---|---|
| Anti-microbial Agent | 10 Minutes of Chewing |
| Menthol | 9% |
| Menthone | 10.4% |
| Methyl Salicylate | 8.9% |
| Eucalyptol | 19.9% |

Chewing gum compositions typically contain a substantially insoluble gum base in amounts typically in the range of 10 to 70% by weight. The gum base retains and therefore does not effectively release typical anti-microbial agents. Accordingly, the factor Q in oral compositions such as chewing gum compositions influences the selection of a suitable value for P. Thus, the germ kill potential (P) may be increased by increasing the concentration of each of the anti-microbial agents, by selecting anti-microbial agents having a higher germ kill rating, by increasing the mass of the product or by a Q value of greater than 1.0. Conversely, the germ kill potential can be reduced by decreasing one or more of the variables mentioned above.

The germ kill potential (P) is a value or range of values providing desirable germ kill properties and acceptable taste characteristics to an oral composition. The germ kill potential (P) may vary depending on the type of oral composition.

The germ kill rating (R) of each anti-microbial agent used in the oral composition is the ratio of the maximum acceptable taste concentration (ATC) of the anti-microbial agent and the minimum inhibitory concentration (MIC). The germ kill rating (R) is therefore a value representative of the suitability of a particular anti-microbial agent to provide a balance between acceptable taste and germ killing efficacy. A relatively high (R) value is indicative of an anti-microbial agent that can be used in a relatively large range of concentrations without adversely effecting taste while still obtaining the desired germ kill properties. A relatively low (R) value indicates that the concentration of the particular anti-microbial agent must be kept in a relatively narrow range so as not to adversely effect the taste of the composition while still maintaining effective germ kill properties.

The selection of a suitable concentration of the anti-microbial agent will be governed in part by the MIC and ATC values. In an oral composition containing a single anti-microbial agent, the actual concentration of the anti-microbial agent must be at least the MIC concentration but not higher than the ATC concentration.

The selection of a suitable concentration for the anti-microbial agent within the range of concentrations will depend on the type of anti-microbial agent, the type of oral composition and the desirable taste characteristics of the oral composition. For example, higher concentrations of the anti-microbial agent within the range of concentrations can be used if a more pronounced taste is desired.

When a composition contains more than one anti-microbial agent, the relative concentrations of each of the anti-microbial agents will in part be governed by the respective (R) values. However, multiple anti-microbial agents may include agents whose (R) value falls outside the MIC or ATC concentration levels. The concentration of the anti-microbial agents may vary over a wide range so long as at least one such anti-microbial agent, preferably more than one, have concentrations between the MIC and ATC concentrations and the (P) value of the composition is a targeted (P) value (i.e. provides a composition having effective germ kill properties and desirable taste characteristics). Thus an anti-microbial agent such as menthol may be added to an oral composition containing multiple anti-microbial agents in a concentration less than the MIC value. In this case, menthol would be used solely for its taste characteristics.

The maximum acceptable taste concentration (ATC) of an anti-microbial agent can be determined through known population sampling methods. Generally, the anti-microbial agent is combined in varying concentrations with a solid carrier to yield test samples. A group of human subjects is selected and asked to taste each of the samples and advise at what concentration the anti-microbial agent begins to exhibit an unacceptable taste. The results are averaged and analyzed according to known statistical methods to yield the ATC value for a particular anti-microbial agent in a given solid carrier (e.g. mint lozenge). Germ kill ratings (R) for representative anti-microbial agents in a mint lozenge having a weight of 1.5 grams are shown below.

TABLE 2

| | Minimum Inhibitory | Maximum Acceptable | |
|---|---|---|---|
| Anti-microbial | Concentration | Taste Concentration | Germ Kill |
| Agent | (MIC) ppm | (ATC) ppm | Rating (R) |
| Cinnamon Oil | 125 | 5,000 | 40 |
| Menthol | 500 | 12,000 | 24 |
| Spearmint | 800 | 12,000 | 15 |
| Peppermint | 1,000 | 12,000 | 12 |
| Thymol | 87 | 100 | 1.1 |

Table 2 provides germ kill ratings for five anti-microbial agents which are essential oils. Cinnamon oil provides the highest germ kill rating (R) (i.e. 40) because the maximum acceptable taste concentration (ATC) is relatively high compared with the minimum inhibitory concentration (MIC). Each of menthol, spearmint and peppermint have higher ATC concentrations but because their respective MIC concentrations are significantly higher than cinnamon oil their germ kill ratings (R) are lower (i.e. 24, 15 and 12, respectively).

Thymol has a very low minimum inhibitory concentration so that it is highly effective in killing microorganisms found in the oral cavity. However, thymol has a very low maximum acceptable taste concentration and therefore provides a germ kill rating (R) of 1.1 which is lower than the other four essential oils in Table 2 even though the other essential oils have a higher minimum inhibitory concentration.

It therefore follows that the selection of an anti-microbial agent based on a particular germ kill rating, the concentration of the anti-microbial agent, the mass of the oral composition and the Q value provides an oral composition with a germ kill potential (P) effective to kill germs yet possess desirable taste characteristics.

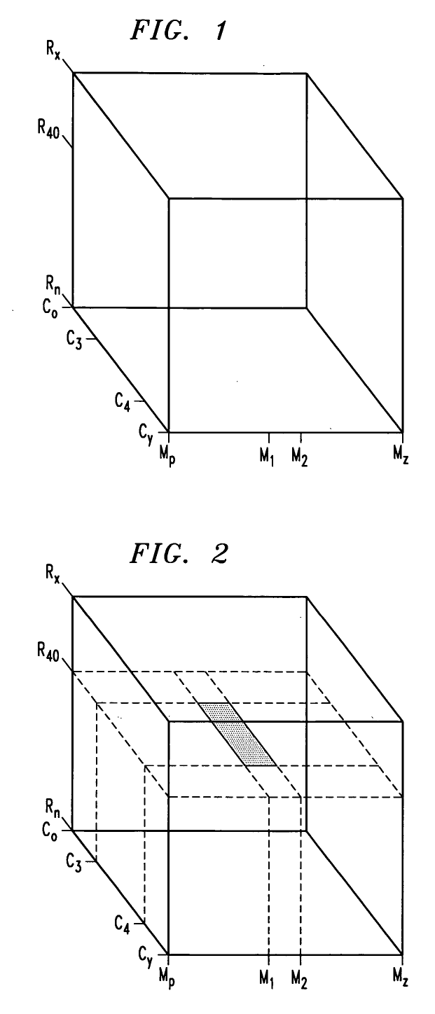

The method of the present invention establishes a germ kill potential value (P) for an oral composition and the selection of at least one targeted anti-microbial agent at an effective concentration which has a germ kill rating suitable for forming an oral composition such as a chewing gum or confection having a particular mass and Q factor. A desirable germ kill potential (P) can be obtained by employing a grid such as shown in FIG. 1. The three dimensional grid plots three variables used in the present method, namely, the germ kill rating of the anti-microbial agent from a low value of Rₙ to a high value of Rₓ, the concentration of the anti-microbial agent from a low value of Cₒ to a high value of C_{y} and the mass of the product from a low value of Mₚ to a high value of M_{z}.

The selection of a suitable (P) value which provides the desired germ kill properties and taste characteristics can be obtained from the grid by separately considering each of the variables. By way of example, consider the production of a stick of gum. The mass of a stick of gum is essentially predetermined and therefore will be within a finite range of mass values within the grid range of Mₚ to M_{z}. As shown in FIG. 2 and by way of example only, the mass of the particular chewing gum stick is in a relatively narrow range from a low value of M₁ to a high value M₂. The mass of the product therefore becomes a limiting factor in reaching a desired (P) value for the product.

If one desires to produce the chewing gum product with cinnamon oil, then the germ kill rating of cinnamon oil needs to be determined. It will be noted that the germ kill rating for a chewing gum product is independently obtained by determining the MIC and ATC values for the chewing gum product as previously described. By way of example, the germ kill rating for the cinnamon oil in the chewing gum product is 40 (shown in FIGS. 1 and 2 as R₄₀). The germ kill rating 40 provides significant flexibility in choosing a desirable concentration of the anti-microbial agent between the values C_{O} and C_{Y}. This is because the maximum acceptable taste concentration is 40 times greater than the minimum inhibitory concentration and therefore one can select a concentration of the anti-microbial between the values C_{O} and C_{Y}. This broad range of concentration levels is indicated in the grid of FIG. 2 represented by the values C₃ and C₄.

By plotting R₄₀, C₃, C₄ and M₁ and M₂ on the grid, the enclosed area within the grid matches a preselected preliminary (P) value or preliminary range of (P) values (i.e. a pre-targeted (P) value).

Once the preliminary (P) value has been obtained, then the (P) value must be adjusted for the factor Q (i.e. the relative rate of release of the anti-microbial agent from the composition into the oral cavity). By way of example, if the preliminary (P) value for a confection containing eucalyptol in a given concentration (e.g. pressed mint) is X and the Q value is 1.0 (i.e. representing 100% release in a fixed period of time such as ten minutes in the oral cavity) the final or targeted (P) value will be the same as the preliminary (P) value (i.e. X).

If the composition is a chewing gum containing eucalyptol in the same concentration and having the same mass, then as shown by the data in Table 1 the targeted (P) value is determined by multiplying the preliminary (P) value X by a Q factor of 5.025 (i.e. 100%/19.9%) to arrive at a targeted (P) value of 5.025X.

The starting targeted (P) value for a particular product can be obtained from a database of known compositions. Thus, the database can provide a range of (P) values determined experimentally through the use of one or more anti-microbial agents. Given that a certain product is to be produced and a desirable (P) value is known, one can employ the grid in the manner shown and described in connection with FIGS. 1 and 2 to produce a product having a desired anti-microbial agent or combination of anti-microbial agents which can be employed in the product in a preselected concentration.

If the (P) value for a given product is not known, then the method of the present invention can be employed to determine a desirable (P) value for the particular product in the following manner. For a given anti-microbial agent, the germ kill rating is determined as described previously and plotted on the grid shown in FIG. 1. Since the mass for the product is typically within a relatively narrow range, a few samples of the product may be prepared with the anti-microbial agent within the desired mass range of the product (e.g. a chewing gum stick has a mass within the range of M₁ to M₂ shown in FIG. 2). Samples are also prepared varying the concentration of the anti-microbial agent. The various samples are tested and those that are determined to have effective germ kill properties and desirable taste characteristics are plotted on the grid to arrive at a preliminary (P) value subject to a determination of the targeted (P) value by multiplying the preliminary (P) value by the Q factor for the particular product. Once the targeted (P) value is obtained, then the method of the invention can be carried out to produce similar products with different anti-microbial agents having different germ kill ratings as previously described, all satisfying the targeted (P) value for the particular product.

One method of formulating the oral composition at the targeted germ kill potential value (P) is to fix all but one of the relevant variables at designated values and then modify the value of a single relevant variable until the targeted germ kill potential value (P) is obtained or an optimum (P) value is obtained. For example, for the production of a stick of gum containing cinnamon oil as the single anti-microbial agent, the Q factor is fixed and R value of the anti-microbial agent is fixed while the concentration is varied within the ATC and MIC values until the targeted germ kill potential value is obtained.

The oral compositions of the present invention in addition to the anti-microbial agent further comprise a carrier in a suitable amount to accommodate the other components of the formulation. The terms “carrier” or “orally acceptable carrier” are meant to encompass orally acceptable vehicles capable of being mixed with the active components for delivery to the oral cavity for cleansing and disinfecting the oral cavity, and which will not cause harm to warm-blooded animals including humans. The carriers include those components of the composition that are capable of being commingled without interaction in a manner which would substantially reduce the composition's stability and/or efficacy for oral cleansing and disinfecting in the oral cavity in warm-blooded animals including humans, in accordance with the compositions and methods of the present invention.

The carriers of the present compositions include one or more compatible solid filler diluents or encapsulating substances, which are suitable for oral administration. The carriers or excipients of the present invention may be in any form appropriate for the mode of delivery, for example, powders, solids and the like, and can include conventional components typically associated with tablets, pressed mints, nougats, wafers, lozenges, hard candies and other confectioneries. Carriers suitable for the preparation of compositions of the present invention are well known in the art. Their selection may also depend on secondary considerations like taste, cost, shelf stability and the like.

Other types of additives or ingredients which may be included in the compositions of the present invention, include, for example, fluoride ion releasing compounds, thickening agents, humectants, flavoring and sweetening agents, anticalculus agents, alkali metal bicarbonate salts, solvents, remineralizers and other additives such as anti-inflammatory agents, and the like. Suitable remineralizers include, for example, calcium phosphate salts such as α-tricalcium phosphate, monocalcium phosphate monohydrate, anhydrous dicalcium phosphate, dicalcium phosphate dihydrate, octacalcium phosphate or tetracalcium phosphate; and calcium glycerophosphate, and combinations thereof.

In one embodiment of the present invention, the anti-microbial agent may be encapsulated to prevent premature degradation and to control the release rate from the composition to further enhance flavor and taste, while maintaining a suitable germ kill rating (R) and germ kill potential (P). Processes for embedding or encapsulating anti-microbial agents such as through microencapsulation to yield beads containing the anti-microbial agent are generally known in the art. The anti-microbial agent may be encapsulated in encapsulating substances including, but not limited to, edible natural or synthetic gums such as xanthan gum or guar gum; edible oils such as peanut oil, coconut oil, palm oil, or safflower oil; polymers such as gelatin, starches, polyamides, polyurethanes, or ethylcellulose; olefinic copolymers such as carbowax; resins; waxes such as paraffin; mineral oils or other edible inert carriers capable of coating and preserving the anti-microbial agent until release such as through mechanical action (e.g. chewing) or by dissolution especially interaction with saliva or water in the mouth. Further details on processes for encapsulating active ingredients including microencapsulation, may be found in U.S. Pat. Nos. 4,867,902; 5,403,578; 5,976,507 and 6,258,343, the contents of each are incorporated herein by reference.

In a more preferred embodiment of the present invention, there is provided a confectionery composition containing at least one essential oil. The preparation of confectionery compositions is well known and has changed very little over the years. Candy confectionery compositions are typically divided into “hard” confectionery and “soft” confectionery compositions. The anti-microbial agents can be employed in the confectionery compositions including hard and soft candy confections in accordance with the method of the present invention.

The term “confectionery or confectionery composition” as used herein includes, but is not limited to: nougats, candies, panning goods, gel confections, fondants, lozenges, mints, troches, pastilles, microcapsules, and other solid forms including freeze dried forms (cakes, wafers, and tablets) and slow dissolving solid forms including compressed tablets and other compositions falling within the generally accepted definition of confectionery compositions.

Hard candy confectioneries may be processed and formulated by conventional means. In general, a hard confectionery has a base composed of a mixture of sugar and other carbohydrate bulking agents kept in an amorphous or glassy condition. This form is considered a solid syrup of sugars generally having from about 0.1% to 1.5% by weight water based on the total weight of the composition. The base typically contains up to about 92% by weight sugar, up to 55% by weight corn syrup, and from about 0.1% to 5% by weight water, based on the total weight of the final composition. The syrup component is generally prepared from sucrose and corn syrups, however it may include other ingredients. Further ingredients such as flavorings, sweetening agents, acidulants, colorants and the like may also be added.

Such confectionery compositions may be routinely prepared by conventional methods such as those involving fire cookers, vacuum cookers, and scraped-surface cookers also referred to as high speed atmospheric cookers as known in the art. The optimum mixing required to uniformly mix the flavoring agent, colorants, and other ingredients during conventional manufacturing of hard confectionery is determined by the time needed to obtain a uniform distribution of ingredients. Typically, mixing times of from about 4 to 10 minutes have been found to be acceptable.

Once the candy mass has been properly tempered, it may be cut into workable portions or formed into desired shapes. A variety of forming techniques may be utilized depending upon the shape and size of the final product desired. A general discussion of the composition and preparation of hard confectioneries can be found in H. A. Lieberman, Pharmaceutical Dosage Forms: Tablets, Volume I (1980), Marcel Dekker, Inc., New York, NY at pages 339 to 469, the content of which is incorporated herein by reference.

The apparatus useful in accordance with the present invention comprises cooking and mixing apparatus well known in the confectionery making arts, and therefore the selection of the specific apparatus will be apparent to the skilled artisan.

In contrast, compressed tablets or pressed mints contain particulate materials and are formed into structures under pressure. These confectioneries generally contain sugars or a water soluble polyhydric alcohol (polyol) such as mannitol, xylitol, sorbitol, maltitol, a hydrogenated starch hydrolysate (“Lycasin”), hydrogenated glucose, hydrogenated disaccharides and/or hydrogenated polysaccharides, in an amount up to 95% by weight based on the total weight of the final composition. Solid salts such as sodium bicarbonate, sodium chloride, potassium bicarbonate, or potassium chloride may totally or partially replace the polyol. Other typical tablet excipients may include, but not limited to, binders, lubricants, flavoring agents, colorants and the like.

Confectionery compositions in the form of pressed tablets such as mints may generally be made by combining finely sifted sugar or sugar substitute, flavoring agent (e.g., peppermint flavor), bulking agent such as gum arabic, and an optional coloring agent. The flavoring agent and bulking agent are combined and then gradually the sugar or sugar substitute are added along with a coloring agent if needed.

The resulting product is then granulated by passing through a sieve of desired mesh size (e.g., 12 mesh) and then dried typically at temperatures of from about 55° C. to 60° C. The resulting powder is fed into a tableting machine fitted with a large size punch and the resulting pellets are broken into granules and then pressed.

Lozenges contain about 2% hydrocolloid as a barrier agent to provide a shiny surface as opposed to a tablet which has a smooth finish. The lozenge or tablet may optionally be coated with a coating material such as waxes, shellacs, carboxymethylcellulose, polyethylene/maleic anhydride copolymer or Kappa-carrageenan, to further increase the time it takes the tablet or lozenge to dissolve in the mouth. The coated tablet or lozenge is slow dissolving, providing a sustained release rate of the active ingredients of about 3 to 5 minutes.

Similar to hard candy confectioneries, soft candy confectioneries may be utilized in the present invention. The preparation of soft confectionery compositions such as nougat involves conventional methods including the combination of two primary components, specifically, (1) a high boiling syrup such as corn syrup and the like, and (2) a relatively light textured frappe, generally prepared from egg albumin, gelatin, vegetable proteins, such as soy derived ingredients, sugarless milk derived ingredients such as milk proteins, and mixtures thereof. The frappe is generally relatively light, and may, for example, range in density from about 0.5 to 0.7 g/cc.

The high boiling syrup, or “bob syrup” of the soft confectionery is relatively viscous and has a higher density than the frappe component, and frequently contains a substantial amount of a carbohydrate bulking agent. Conventionally, the final nougat composition is prepared by the addition of the “bob syrup” to the frappe under agitation, to form the basic nougat mixture. Further ingredients such as flavoring, additional carbohydrate bulking agent, colorants, preservatives, and the like may be added thereafter under agitation. A general discussion of the composition and preparation of nougat confectioneries may be found in B. W. Minifie, Chocolate, Cocoa and Confectionery: Science and Technology, 2^{nd} Edition, AVI Publishing Co., Inc., Westport, Conn. (1980), at pages 424-425, the content of which is incorporated herein by reference.

The procedure for preparing the soft confectionery involves known procedures. In general, the frappe component is prepared first and thereafter the syrup component is slowly added under agitation at a temperature of at least 65° C., preferably at least 100° C. The mixture of components is continued to be mixed to form a uniform mixture, after which the mixture is cooled to a temperature below 80° C., at which point, the flavoring may be added. The mixture is further mixed for an additional period until it is ready to be removed and formed into suitable confectionery shapes.

The present invention is further directed to methods for preparing the consumable oral compositions of the present invention. The anti-microbial agents may be incorporated into an otherwise conventional hard or soft confectionery composition using standard techniques and equipment known to those skilled in the art. The apparatus useful in accordance with the present invention comprises mixing and heating apparatus well known in the confectionery manufacturing arts, and therefore the selection of the specific apparatus will be readily apparent to the skilled artisan.

In the present method, a consumable oral composition is made by admixing an anti-microbial agent having a suitable germ kill rating (“R”) into the confectionery along with other ingredients to provide a final composition having a desired germ kill potential (P) value. Other ingredients may be added to the composition as required by the nature of the desired composition as well known by those having ordinary skill in the art. The final compositions are readily prepared using methods generally known in the food technology and pharmaceutical arts. Thereafter the confectionery mixture may be formed into desirable confectionery shapes.

The consumable oral compositions may be formulated with conventional ingredients, which offer a variety of textures to suit particular applications. Such ingredients may be in the form of hard and soft confectioneries, tablets, toffee, nougat, chewy candy, chewing gum, and the like, both sugared and sugarless. Suitable ingredients may be selected from a range of materials including, but are not limited to, diluents, binders, adhesives, lubricants, disintegrants, bulking agents, humectants, buffers and adsorbents. The preparation of such confectionery compositions is well known.

Further details regarding the preparation of confectionery compositions can be found in Skuse's Complete Confectioner (13^{th} Edition) (1957) including pp. 41-71, 133-144, and 255-262; and Sugar Confectionery Manufacture (2^{nd} Edition) (1995), E. B. Jackson, Editor, pp. 129-168, 169-188, 189-216, 218-234, and 236-258 each of which is incorporated herein by reference.

The present invention is also directed to a method for reducing the presence of microorganisms in the oral cavity, comprising administering to the oral cavity of a warm-blooded animal including humans an effective amount of the consumable oral composition of the present invention. The present consumable oral compositions of the present invention is preferably administered, applied or contacted to the surfaces of the teeth and oral cavity for a sufficient time to kill microorganisms thereon, in one or more conventional ways.

The consumable oral composition of the present invention in the form of a confectionery may be wholly or partially consumed during the period of time that the composition is retained in the mouth of the consumer such as by chewing or sucking. Suitable effective periods of time may range from 5 seconds to 5 minutes. The frequency of the application or contact of the composition with the teeth and surfaces of the oral cavity is preferably from about once a week to about four times per day, more preferably from about 3 times per week to three times per day, even more preferably at least once per day. The period of such treatment typically ranges from about one day to a lifetime.

The forgoing discussion discloses and describes merely exemplary embodiments of the present invention. One skilled in the art will readily recognize from such discussion, and from the accompanying claims, that various changes, modifications, and variations can be made therein without departing from the spirit and scope of the invention as defined in the following claims.

### EXAMPLES 1-3

### Clinical Study of Consumable Oral Compositions Containing Essential Oils

Three consumable oral compositions containing essential oils were prepared in the form of pressed mints to test their bacteria reducing potency. Each composition contained at least one of the following essential oils: spearmint, menthol, and peppermint. The germ kill potential (P) for each consumable oral composition was calculated using its mass and the concentrations of the corresponding essential oils listed in Table 3 in combination with the corresponding germ kill ratings (R) of the essential oils listed in Table 2.

TABLE 3

| Compositions | Spearmint | | Menthol | | Peppermint | Mass |
|---|---|---|---|---|---|---|
| Consumable oral | 0.45 | pph* | 0.55 | pph | — | 1.0 g |
| composition 1 | | | | | | |
| Consumable oral | — | | 0.55 | pph | 0.68 pph | 1.0 g |
| composition 2 | | | | | | |
| Consumable oral | 1.0 | pph | 1.2 | pph | — | 0.3 g |
| composition 3 | | | | | | |

*pph = parts per hundred

The germ kill potential (P) for consumable oral composition 1 was calculated by taking the sum of the products of the concentration of the essential oil and the germ kill rating of the essential oil. The resulting sum was multiplied by the mass of the consumable oral composition (1 g) and a Q factor of 1.0 (representing immediate release of the anti-microbial agent) to yield a targeted germ kill potential (P) of about 19.95 as shown below.

$$\begin{matrix}{( P )}_{Consumable oral composition 1} = [ ( {Conc}_{Spearmint} \times {R}_{Spearmint} ) + \\ ( {Conc}_{Menthol} \times {R}_{Menthol} ) ] \times Mass \times Q factor \\ = [ ( 0.45 \times 15 ) + ( 0.55 \times 24 ) ] \times 1 \times 1.0 \\ = 19.95\end{matrix}$$

The calculations of the germ kill potential (P) for consumable oral compositions 2 and 3 were carried out in the same manner as consumable oral composition 1. The germ kill potential (P) for each of the compositions 2 and 3 is listed in Table 4 below.

TABLE 4

| Compositions | Germ Kill Potential (P) |
|---|---|
| Consumable oral | 19.95 |
| composition 1 | |
| Consumable oral | 21.36 |
| composition 2 | |
| Consumable oral | 13.14 |
| composition 3 | |

A randomized observer-blind crossover study was implemented to determine the anti-microbial efficacy of the consumable oral compositions 1-3 on sulfur generating bacteria populations found on the surface of the tongue. The study was conducted with 40 human subjects to compare the anti-microbial activity of the three consumable oral compositions over a control (i.e., no treatment). The population of the sulfur generating bacteria was measured before administration of the consumable oral compositions, and was measured again 30 minutes after the compositions had dissolved in the mouth of the human subjects.

A baseline measurement was made for each subject by plating oral bacteria taken from the subjects' mouth with a tongue swab onto a selective agar medium. Subjects were assigned a sequence that specified the order of active treatments or no treatments. Data was collected and tabulated to obtain the following results listed in Table 5.

TABLE 5

| Oral Compositions | % Reduction in Bacteria Flora |
|---|---|
| Consumable oral | 58.6% |
| composition 1 | |
| Consumable oral | 46.9% |
| composition 2 | |
| Consumable oral | 17.8% |
| composition 3 | |

### EXAMPLE 4

From the data shown in Examples 1-3 and particularly Table 4 and Table 5, it was determined that a germ kill potential (P) in the range of 19.5 to 21.5 would be desirable for a pressed mint product having a mass of one gram and containing spearmint (germ kill rating of 15) as an anti-microbial agent. Given the mass of one gram, the Q factor of 1.0 and a germ kill rating of 15.0, a concentration of about 1.3 to 1.43 pph is selected to provide the desired germ kill potential (P).

### EXAMPLE 5

### A Chewing Composition Containing Menthol

A chewing gum slab composition containing menthol as the anti-microbial agent is prepared having the following characteristics, a mass of 1.8 grams, a concentration of menthol of 3% (3 pph), and a Q factor of 11.1 (100%/9%, see Table 1). The germ kill rating of menthol is 24. The targeted (P) value is determined as follows:

$$\begin{matrix}{( P )}_{Chewing gum composition 1} = ( {Conc}_{Menthol} \times {R}_{Menthol} ) \times Mass \\ = [ ( 3 \% \times 24 ) \times 1.8 \times 11.1 \\ = 14.38\end{matrix}$$

1. A method of formulating an oral composition containing at least one anti-microbial agent to obtain said oral composition having acceptable germ kill properties and taste characteristics, comprising:
a) establishing a targeted germ kill potential value (P) for the oral composition;
b) selecting at least one anti-microbial agent for the oral composition based on the germ kill rating and concentration of the anti-microbial agent, the Q factor of the anti-microbial agent in the oral composition and the mass of the oral composition which exhibits the targeted gum kill potential (P); and
c) formulating the oral composition using said at least one anti-microbial agent.

2. The method of claim 1 wherein the step of establishing a germ kill potential comprises;
a) determining a preliminary germ kill potential for the composition; and
b) adjusting the preliminary germ kill potential by the Q factor to obtain the targeted germ kill potential.

3. The method of claim 1 wherein the preliminary germ kill potential [P-preliminary] is determined by the formula
$$P ( preliminary ) = ( {\sum }_{n = 1}^{Y} {concentration}_{n} \times {R}_{n} ) \times Mass$$
Mass=the mass of the oral composition capable of delivering the anti-microbial agent to the oral cavity,
concentration=the concentration of the anti-microbial agent measured in parts per hundred,
R=the germ kill rating of the anti-microbial agent, and
Y=number of anti-microbial agents in the oral composition.

3. (canceled)

4. The method of claim 1 wherein the step of establishing a germ kill potential comprises;
a) determining a maximum acceptable taste concentration (ATC) for the anti-microbial agent in the oral composition,
b) determining a minimum inhibitory concentration (MIC) of the anti-microbial agent in the oral composition, and
c) dividing the ATC by the MIC to obtain the germ kill rating.

5. The method of claim 1 wherein the at least one anti-microbial agent comprises at least one essential oil.

6. The method of claim 5 wherein the essential oils are selected from the group consisting of menthol, eucalyptol, thymol, cinnamon oil, peppermint and spearmint.

7. The method of claim 1 wherein the oral composition releases all of the anti-microbial agent within a fixed period of time in the oral cavity and the corresponding Q factor is 1.

8. The method of claim 1 wherein the oral composition is a confectionery composition.

9. The method of claim 1 wherein the oral composition is a chewing gum composition.

10. The method of claim 9 wherein the Q factor is greater than 1.0.

11. The method of claim 1 comprising selecting the anti-microbial agent by establishing fixed values for all but one of the variables selected form the group consisting of the germ kill rating, the concentration of the anti-microbial agent, the mass of the composition and the Q factor of the composition and adjusting one of the variables until the targeted germ kill potential value (P) is obtained.

12. The method of claim 11 wherein the step of adjusting one variable comprises adjusting the concentration of the anti-microbial agent.

13. The method of claim 1 wherein the step of establishing the targeted germ kill potential value for the composition comprises:
1) producing a series of samples containing the at least one anti-microbial agent, each sample having a concentration of the anti-microbial agent between a minimum inhibitory concentration and a maximum acceptable taste concentration and a preselected mass,
2) obtaining the germ kill rating for the anti-microbial agent,
3) determining the Q factor for the anti-microbial agent in the composition,
4) determining the (P) value for each sample, and
5) determining a targeted (P) value from the (P) values of the individual samples.

14. The method of claim 12 wherein the step of determining the Q factor comprises measuring the amount of the anti-microbial agent present in the oral composition before placement in the oral cavity and the amount of the anti-microbial agent present in the oral composition after being in the oral cavity for a fixed period of time.

15. The method of claim 1 wherein the step of establishing a targeted germ kill potential value comprises preparing a series of samples of the oral composition having variable anti-microbial agents and concentrations for a fixed mass for the oral composition, providing the samples to human subjects to determine if the sample has a desirable taste characteristic, testing the samples to determine if they are effective in killing microorganisms in the oral cavity and identifying targeted samples that provide both desirable taste characteristics and germ kill properties, and determining the targeted germ kill potential value from the targeted samples.

16. The method claim 1 wherein Step (c) comprises:
1) plotting an area on a graph comparing the germ kill rating of the anti-microbial agent, a range of concentrations of the anti-microbial agent and a range of masses for the oral composition and the Q factor; and comparing the plotted area representing the targeted germ kill potential value.

17. The method of claim 16 further comprising:
observing where the plotted area of the graph matches with the targeted germ kill potential value to identify a matched graph portion and selecting an oral composition which meets the criteria of the matched graph portion.

18. A method of determining a targeted germ kill potential value (P) for an oral composition containing at least one anti-microbial agent, said oral composition having acceptable germ kill properties and taste characteristics as a result of having such targeted germ kill potential value (P), said method comprising:
a) selecting at least one anti-microbial agent for the oral composition based on a set of variables including germ kill rating and concentration of the anti-microbial agent, the Q factor or the anti-microbial agent in the oral composition and the mass of the oral composition which exhibits the targeted gum kill potential (P);
b) formulating the oral composition using said at least one anti-microbial agent into a series of samples varying at least one of said variables; and
c) testing said samples to determine which samples exhibit acceptable taste characteristics and acceptable gum kill properties.

19. The method of claim 18 wherein the step of testing the samples comprises having human subjects ingest the samples and evaluate the taste characteristics of the samples.

20. The method of claim 18 comprising:
1) producing a series of samples containing the at least one anti-microbial agent, each sample having a concentration of the anti-microbial agent between a minimum inhibitory concentration and a maximum acceptable taste concentration and a preselected mass,
2) obtaining the germ kill rating for the anti-microbial agent,
3) determining the Q value for the anti-microbial agent in the composition,
4) determining the (P) value for each sample, and
5) determining a targeted (P) value from the (P) values of the individual samples.

21. The method of claim 18 wherein the step of establishing a targeted germ kill potential value comprises preparing a series of samples of the oral composition having variable anti-microbial agents and concentrations for a fixed mass for the oral composition, providing the samples to human subjects to determine if the sample has a desirable taste characteristic, testing the samples to determine if they are effective in killing microorganisms in the oral cavity and identifying targeted samples that provide both desirable taste characteristics and germ kill properties, and determining the targeted germ kill potential value from the targeted samples.

22. The method of claim 18 further comprising:
1) plotting an area on a graph comparing the germ kill rating of the anti-microbial agent, a range of concentrations of the anti-microbial agent and a range of masses for the oral composition, to determine the targeted germ kill potential value.

23. The method of claim 1, further comprising:
establishing a germ kill rating (R) for the at least one anti-microbial agent.
